(19) United States
(12) Patent Application Publication
(10) Pub. No.: US 2023/0218058 A1
(43) Pub. Date: Jul. 13, 2023

(54) ROBOTIC DEBRIDEMENT AND PERFORATING OF NAILS BASED ON SURFACE AND/OR THICKNESS SCANNING APPARATUS

(71) Applicant: RAPHA ROBOTICS INCORPORATED, Lexington, MA (US)

(72) Inventor: Elad Taig, Lexington, MA (US)

(73) Assignee: RAPHA ROBOTICS INCORPORATED, Lexington, MA (US)

(21) Appl. No.: 17/615,328

(22) PCT Filed: Sep. 15, 2021
(86) PCT No.: PCT/US21/50437

§ 371 Date: Nov. 30, 2021

Related U.S. Application Data
(60) Provisional application No. 63/079,945, filed on Sep. 17, 2020.

(51) Int. Cl. A45D 29/17; A61L 2/10; A61L 2/24

(52) U.S. Cl. CPC A45D 29/17; A61L 2/10; A61L 2/24; A61L 2202/14; A61L 2202/11; A61L 2202/24

(57) ABSTRACT

The present disclosure features systems and devices for robotic debridement and perforating of finger- and toenails, as well as methods of use of the same. The systems and devices may be used for in the treatment of fungal infection of the nail, as well as for cosmetic uses, e.g., pedicure and manicure.

### CROSS-REFERENCE TO RELATED APPLICATIONS

This application is a National Stage Application filed under 35 U.S.C. 371 based on International Patent Application No. PCT/US2021/050437, filed on Sep. 15, 2021, which claims the priority of U.S. Provisional Application No. 63/079,945, filed Sep. 17, 2020, both of which are incorporated herein by reference in their entirety.

### GOVERNMENT LICENSE RIGHTS

This invention was made with government support under Grant Number 2126854 awarded by the National Science Foundation (NSF). The government has certain rights in the invention.

### FIELD OF THE INVENTION

The present invention relates to systems, e.g., medical devices, control systems and methods for robotic debridement and perforating of nails based on scanning of the nail's surface and/or thickness. More particularly, the systems and methods are useful in the treatment of Onychomycosis (a fungal infection of the nails). The present invention also relates to use of the systems and methods for nail polishing and filing for manicure and pedicure.

### BACKGROUND

Onychomycosis, also known as tinea Unguium, is a fungal infection of the nail. Symptoms may include white or yellow nail discoloration, thickening of the nail, and separation of the nail from the nail bed. Toenails or fingernails may be affected, but it is more common for toenails to be affected. A number of different types of fungus can cause onychomycosis including dermatophytes and Fusarium. The antifungal medication, terbinafine, taken by mouth appears to be the most effective but is associated with liver problems. There is a ciclopirox-containing nail polish, but it does not work as well. The recurrence rate of infection is up to 50% following any of the above-mentioned treatments. It occurs in about 10 percent of the adult population. Males are affected more often than females. In immuno-compromised people (e.g., HIV positive, chemotherapy patients, etc.) it can lead to serious systemic infections.

Onychomycosis is not merely a cosmetic problem, as it negatively affects patients' emotional, social, and occupational functioning, e.g., embarrassment in social situations, employer's reluctance to employ onychomycosis patients in jobs that require food handling or contact with the public.

Present treatments for onychomycosis include administering systemic and/or local antifungal therapy, e.g., the antifungal medication terbinafine, surgical nail removal and electromagnetic radiation therapy.

Topical administration of antifungal drugs is proven to be largely inefficient in clearing the infection due to its limited ability to penetrate the nail plate.

Systemic oral antifungal therapy has been seen to be more effective in treating onychomycosis than topical treatments. Commonly used antifungals, such as itraconazole and terbinafine, have shown cure rates from 50% to 80%. However, recurrence rate in studies monitoring up to 3 years of treatment or post-treatment of such oral antifungal drugs remained high: 3%-20% for terbinafine and 21%-27% for itraconazole. Because of their systemic and long-term administration, care should be taken to avoid toxic side effects, which in serious cases can include liver toxicity and heart failure.

In recent years there has been an increase in device-based onychomycosis therapy. Many of these therapies use electromagnetic radiation aimed at destroying the infective fungus. Some of these devices utilize wavelengths which are at least partially transmitted through the nail and then absorbed in the infected nail bed. Other devices utilize wavelengths that are strongly absorbed at the nail surface and do not get substantially transmitted to the nail bed. This absorption at the nail surface leads to the release of heat at the nail surface, which is diffused through the nail to the underlying nail bed, causing a temperature rise within the nail bed. Laser energy is delivered in the amount necessary to heat the whole thickness of the nail and the upper surface of the nail bed to temperatures that induce death of the infecting organism.

Irradiating the nail with UV light is another such approach. While UV irradiation works well in eliminating the infectious microorganisms, it can damage the underlying tissue and surrounding skin and is also a known mutagen.

Typically, laser wavelengths which are not strongly absorbed by the nail are also not strongly absorbed in the infecting fungi. This results in non-specific bulk heating of the finger, which causes pain and thermal damage to healthy tissue lying deeper.

Fingernails have an average thickness of 0.6 mm in males and 0.5 mm in females. Toenails are thicker, 1.65±0.43 mm and 1.38±0.2 mm in males and females, respectively. Onychomycosis might significantly increase the thickness of the nail plate.

The energy required to cut the nail plate in the longitudinal direction (6 kJ/m²) is 2-fold higher than to cut it in the transverse direction (3 kJ/m²).

A normal, healthy, nail plate has between 25 and 27 layers of dehydrated human skin, keratin. Typically, nail fungi proliferate in the space between the nail plate and the skin below (nail bed). In order to kill toenail fungus or fingernail fungus via electromagnetic irradiation or UV irradiation, a medical laser must penetrate the nail plate without harming the underlying nail plate layers and then break apart the fungus cell structure without burning nerve endings and skin cells located on the nail bed. Current laser-to-heat treatments are not ideal because: (1) the heat can burn the underlying normal nail plate or surrounding skin; (2) attenuation depth, also referred to as therapeutic penetration depth of heat cannot be controlled effectively because heat loses its kill power effectiveness as different layers are encountered; (3) nail fungus located deeper than the minimum effective threshold of heat power remains untreated; and (4) because of inefficient conversion of laser photon quantum energy to thermo energy, current medical lasers are rated at between 4 watts and 80 watts to kill nail fungus. To place in perspective, in the metal fabrication industry, lasers with wattages of between 10 watts and 100 watts are used to cut thick steel plates.

There is a need for improvement in treatment efficacy, e.g., in order to get treatments to effectively penetrate the nail plate and attack the underlying fungus without wasting energy or causing additional pain or damage to the surrounding tissue. There have been hand-debridement techniques used prior to treatment. Debridement of the infected nail removes some infected layers of the nail plate such that a topical or laser treatment can more effectively reach the infected nail bed. Debridement is labor intensive and error prone work. Precise, uniform, and accurate depth control (number of layers removed) during debridement are only achievable using robotic polishing.

Manual debridement, with its limited capabilities, has proven advantages in the treatment of nail fungus. There is a need for more precise, uniform, accurate, and clean debridement method, e.g., through robotic debridement.

The automatic robotic debridement device provides an immediate cosmetic improvement, decreases the fungal mass, increases the efficiency of laser, topical drugs, and other therapies, and prevent reinfection. In concert, this would significantly improve the success rates and reduce recurrence rates which would contribute to a better patient treatment outcome.

### SUMMARY

The present disclosure features a system and method of treatment of nail fungus (onychomycosis), wherein the nail thickness is precisely reduced by robotic debridement prior to applying a laser or topical treatment. In some embodiments, said system comprises a software, control system and a robotic debridement device. In some embodiments, a debridement toolpath is determined by scanning the nail plate using a distance and/or thickness sensor and utilizing a proprietary software and control system that automatically generates the toolpath and controls the robotic debridement tool. The debrided nail thickness is precisely controlled by the robotic debridement device to provide adequate protection to the nail bed while minimizing the required penetration depth or thermal barrier for the electromagnetic radiation.

More specifically, said systems of the present invention may further comprise a proprietary software program which generates an automated toolpath for the debridement tool to follow to debride the nail in a uniform manner.

Embodiments of the disclosure are discussed in detail below with respect to treatment of human finger and toenails. However, in some embodiments, the present disclosure can be employed for treatment of other mammals without limitation. In some embodiments, examples of mammals can include, but are not limited to, domestic and farm animals, such as dogs, cats, and pigs. The present disclosure provides embodiments of systems, devices, and methods of use of the same with respect to treatment of onychomycosis. However, in some embodiments, said systems, devices, and methods of use are used in the treatment of other medical and/or cosmetic conditions without limitation. For example, fungal infection may further include subungual onychomycosis, wherein the fungus spread from the skin of the finger to the nail bed, and candidal onychomycosis, wherein the main source of infection is yeast.

In another aspect, the present disclosure provides a method for precisely drilling holes in the nail plate such that topical antifungal medication can be applied, through these holes, to an infected nail area. In some embodiments, the location and pattern of the drilled holes is manually defined. In other embodiments, the location and pattern of the drilled holes is automated by the software, e.g., utilizing images from a camera and image processing system.

In another aspect, the present disclosure provides a method for polishing and filing a nail for manicure or pedicure. In some embodiments, the polishing toolpath is automatically generated based on a scan of the nail plate using a distance and/or thickness sensors.

The present disclosure further provides that said systems in use for treating an infected nail comprise a robotic debridement and perforating device for use in the robotic debridement and/or perforating of said nail. In some embodiments, said systems further comprise a software and a control system. In some embodiments, said device includes a base, a hand or foot holder, a multi axis motion stage, a debridement head, a distance and/or thickness sensor, and a camera. In some embodiments, said hand or foot holder includes a mechanism to secure individual nails in varying directions during the debridement process. In some embodiments, said multi axis motion stage provides motorized movement for the debridement head, sensors, and camera. In some embodiments, the software and control system scans the nail using the distance and/or thickness sensor and generates a toolpath for the robotic debridement device. In some embodiments, the toolpath is designed in such a way to prevent damage to the surrounding tissues. In some embodiments, wherein a thickness sensor is used, said toolpath is designed in such a way to provide an optimally uniform nail thickness. In some embodiments, the camera of the device provides a view of the nail and of the debridement tool. In some embodiments, using an image processing technique as described herein, the camera can be used to generate limits for the debridement toolpath to prevent any damage to surrounding tissues. In some embodiments, during operation the camera allows the operator to see the debridement tool and stop the machine if needed.

Additional features of the systems, devices and methods of use thereof are described herein in the Detailed Description, Examples, Figures, and Claims.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 is an image of a toenail suffering from a common form of Onychomycosis.

FIG. 2. is a depiction of a nail structure. The nail consists of the nail plate 201, the hard part of the nail made of translucent keratin protein, the underlying bone 205, the nail bed 202, the skin beneath the nail plate 201, the nail matrix (root) 203 and 204, nail folds 206, and the cuticle (eponychium) 208.

FIG. 3 is a depiction of an exemplary treatment system for Onychomycosis comprising the following steps: scanning the nail using the distance and thickness sensors; debridement of the entire infected nail; drilling a predefined pattern and density of tiny holes across the nail plate; eradicating fungus using an exemplary laser treatment; and applying topical drugs, as described herein.

FIG. 4 is a depiction of topical drug penetration through the nail plate and how to calculate the rate of topical drug penetration using Fick's law.

FIG. 5 is a depiction of laser transmission across the nail plate and how to calculate the transmittance using Lambert-Beer's law.

FIG. 6 is an image of a toenail suffering from a common form of Onychomycosis (fungal infection) in a particular region of the nail, as shown by decolorization typical for Onychomycosis, and a pattern of small holes drilled across the infected region.

FIG. 7A and FIG. 7B are exemplary depictions of the robotic debridement device, wherein said device 700 comprises: a base 704, a four sided case 706, a top cover 701, a foot or hand holder 703 and 705, a port in the case to allow the foot/hand holder to enter the case 702, a multi axis motion stage 707, a debridement head 708 a debridement bit 709, a distance/thickness sensor 710, and a camera 711. In some embodiments, said device further includes a low-power laser.

FIG. 8A is a depiction of an array of exemplary debridement and drilling bits.

FIG. 8B is a depiction of an exemplary drilling bit in a bit holder.

FIG. 9 is an exemplary depiction of a force sensor 901 mounted between the drill bit holder 902 and the linear motor 903, wherein the linear motor is mounted on the vertical axis 904 of a multi axis motion stage.

FIG. 10 is an exemplary depiction of a computer and control system in communication with the physical components of the robotic debridement device.

FIG. 11 is an exemplary depiction of a path planning algorithm which would be used by the controller system to create a toolpath for the robotic debridement device.

FIG. 12 is a depiction of three possible two-dimensional plane-filling tool paths.

FIG. 13 is an exemplary depiction of mapping a two-dimensional toolpath on to the nail's three-dimensional surface.

FIG. 14 is an exemplary depiction of the controller system architecture.

### DETAILED DESCRIPTION OF THE DRAWINGS

Unless otherwise defined, all technical and scientific terms used herein have the same meaning as commonly understood by one of ordinary skill in the art to which this invention belongs. In case of conflict, the present application, including definitions will control.

While various embodiments of the invention are shown and described herein, it will be obvious to those skilled in the art that such embodiments are provided by way of example only, and that numerous variations or substitutions may occur to those skilled in the art without departing from the invention. Various alternatives to the embodiments of the invention described herein may be employed.

As used herein, the singular forms “a,” “an,” and “the” include plural references unless the context clearly dictates otherwise. Any reference to “or” herein is intended to encompass “and/or” unless otherwise stated.

In this disclosure, “treatment”, “treat”, and the like refer to but not limited to improving or eliminating a condition, symptoms, disorder, and abnormal appearing.

The term “topical drug”, “anti-fungal drug” and the like as used herein, generally refers to any anti-fungal drug, approved by the US Food and Drug Administration or otherwise, for the treatment of Onychomycosis. Examples of anti-fungal drug can include, but are not limited to, Ciclopirox 8% nail lacquer, Efinaconazole 10% solution, and Tavaborole 5% solution.

The term “laser”, “medical laser”, “laser therapy” and the likes as used herein, include but not limited to, carbon dioxide laser, long Nd:YAG 1064 nm, short-pulsed Nd:YAG, and laser diode 870 nm or 930 nm.

The term “distance sensor”, “laser distance sensor”, “proximity sensors” and the likes as used herein, generally refers to any technology or sensor able to measure the distance to a nearby object with or without a physical contact. Examples of distance sensors type, and technologies can include, but are not limited to, laser sensors, ultrasonic sensors, Infrared sensors, LiDAR, low-coherence interferometry (LCI), time-of-flight sensors, triangulation sensors, and phase-modulation sensors.

The term “thickness sensor” and the likes as used herein, generally refers to any technology or sensor able to measure the thickness of an object without a physical contact. Examples of thickness sensors type, and technologies can include, but are not limited to, laser sensors, ultrasonic sensors, capacitive, low-coherence interferometry (LCI), and eddy current. Embodiments of the disclosure are discussed in detail below assuming a thickness sensor is implemented in the device. However, the disclosed embodiments can be employed with or without a thickness sensor.

The term “force sensor”, “Torque sensor”, and the likes as used herein, generally refers to any sensor or method able to measure force or torque, in one or more axis. Example of force sensors can include, but are not limited to, stain gauge, load cell, and capacitive. Example of torque sensors can include, but are not limited to, strain gauge, torque cell, torque transducer, and electrical motor current.

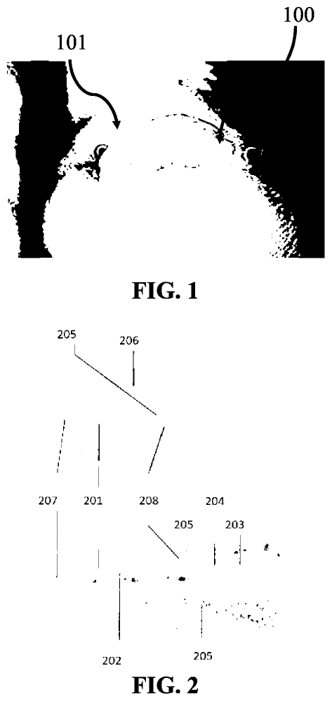

FIG. 1 is an exemplary depiction of a toenail 100 suffering from a common form of Onychomycosis (fungal infection). The toenail 100 include a nail plate 101 showing typical Onychomycosis symptoms, e.g., white, or yellow nail discoloration, thickening of the nail, and separation of the nail plate from the nail bed.

FIG. 2 is an exemplary depiction of the nail structure. A nail is a claw-like keratinous plate at the tip of the fingers and toes in most primates. The nail consists of the nail plate 201, the hard part of the nail, made of translucent keratin protein. Several layers of dead, compacted cells cause the nail to be strong but flexible. Its (transverse) shape is determined by the form of the underlying bone 205. The nail bed 202, the skin beneath the nail plate 201. The nail matrix (root) 203 and 204, located beneath the skin behind the nail and extends several millimeters into the nail itself. The nail matrix produces cells that become the nail plate. As new nail plate cells are made, they push older nail plate cells forward. The nail folds 206 and 205, is the skin that frames each of your nails on three sides. The Cuticle (eponychium) 208, located between the skin of the nail and the nail plate fusing these structures together and providing a waterproof barrier. The Lunula 205, the whitish, half-moon shape at the base of your nail. Each of these structures has a specific function, and if disrupted can result in an abnormal appearing nail.

The present disclosure features systems, devices, and methods of use of the same for debridement or perforating fingers or toenails which are infect with nail fungus, e.g., onychomycosis. In some embodiments, said systems, devices, and methods of use of the same for automatically performing pedicure and manicure for aesthetic purposes. In some embodiments, said systems, devices and methods of use comprise a robotic nail debridement and a perforating device. More specifically, said systems of the present invention may further comprise a proprietary software program which generates a toolpath for the debridement tool to follow to debride the nail in a uniform manner.

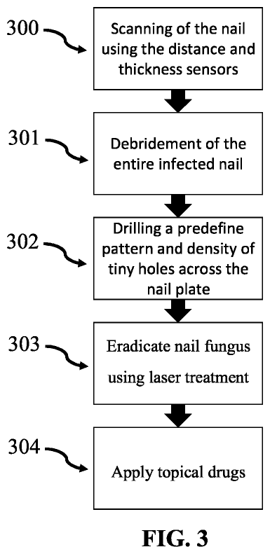

FIG. 3 is an exemplary depiction of a treatment system for Onychomycosis that can include a few steps. In the first step 300, the robotic debridement device is scanning the nail using the distance and thickness sensors. Based on this scan the proprietary algorithm is generating a toolpath for the debridement head. In the second step 301, the robotic debridement device precisely removing infected nail layers from the entire nail plate surface, reducing the nail plate thickness down to its structural yet safe limit. One or more of the following steps could be performed in any required order. In the third step 302, a predefined pattern and density of tiny holes are drilled across the entire nail plate, or only in infected areas. The infected area/s could be defined manually or automatically defined by the software based on images captured by the camera, or information capture by the thickness sensor during the first step 300. In the fourth step 303, a low-power medical laser is used to eradicate the nail fungus. The laser can cover the entire nail plate surface, or only predefined areas. The software can automatically adjust the power or shut down the laser when moving above a drilled hole in the nail plate. In the fifth step 304, an anti-fungal topical drug is applied on the nail plate. The topical drug can easily diffuse through the thin nail plate and can also directly reach the nail bed through the drilled holes. The above steps could be performed in sequence or separately on one or more nails. Furthermore, some or all the steps above could be performed on a single nail at a time or on multiple nails in parallel, e.g., the debridement could be performed on several nails in parallel.

### System and Methods:

The present disclosure features a system for robotic debridement and perforating of fingers or toenails, and methods of use of the same. In some embodiments, said system comprises a physical device for debridement and perforating of a finger or toenail. Physical components of these systems and methods of use will be described herein in greater detail. In some embodiments, said system further comprises a controller system for providing control over the robotic debridement and perforating device. More specifically, said controller system may further comprise a proprietary software. In some embodiments, said proprietary software consists of a novel scan-based conformal debridement algorithm. In some embodiments, said software algorithm comprises program sequences which enable it to take a scan of an uneven nail surface and determine an appropriate toolpath for the debridement head to follow to create an ideal, evenly conforming nail which is then more susceptible to treatment. In some embodiments, said controller system generates motion limits based on the scan previously done. Said limits may prevent damage to surrounding tissue. In some embodiments, said controller system utilizes a distance sensor of the device as described below in order to determine the stopping point of the automated toolpath for the debridement head. Further methods of use of the software components and physical components of the system are described in more detail below.

The robotic debridement device precisely removing infected nail layers, reducing the nail plate thickness down to its structural yet safe limit. The removed layer thickness may be at-least about 0.005 millimeters (mm), 0.01 mm, 0.015 mm, 0.02 mm, 0.025 mm, 0.03 mm, 0.035 mm, 0.04 mm, 0.05 mm, 0.055 mm, 0.06 mm, 0.065 mm, 0.07 mm, 0.075 mm, 0.08 mm, 0.085 mm, 0.09 mm, 0.095 mm, 0.01 mm or higher.

The removed infected nail layers, decreases the fungal mass and make the nail bed more prone to contact with antifungal drugs. The remaining thin nail plate layer continues to protect the nail bed from reinfection.

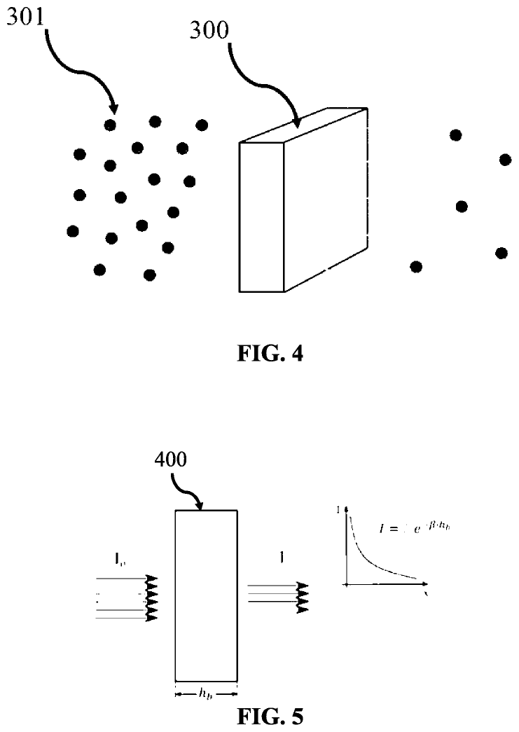

The topical drug penetration rate through the nail plate barrier is inversely proportional to the nail plate thickness, and could be estimate according to Equation 1 (i.e., Fick's law), see FIG. 4:

$$\frac{dM}{dt} = \frac{1}{{h}_{b}} \cdot ( {D}_{B} \cdot A \cdot {C}_{BD} ) \qquad ( 01 )$$

where

$$\frac{dM}{dt}$$

is the amount of topical drug penetrating per unit time through the nail plate 400,
h_{b} is the thickness of the nail plate 400,
D_{B} is the effective diffusion coefficient through the nail plate,
A is nail plate area, and
C_{BD} is the topical drug concentration above the nail plate 401.

According to equation 1, a reduction of the barrier thickness from h_{b} to h_{bf} results in an increase of the penetration rate by a factor of

$$\frac{{h}_{b}}{{h}_{bf}} .$$

For example, a reduction of the nail plate's thickness from 1.65 mm to 0.075 mm results in an increase of penetration rate by a factor of 22, meaning a significant amount of topical drug will penetrate the nail plate in a much shorter amount of time, resulting in greater topical drug efficacy.

Currently, among other confounding factors, laser treatment of nail fungus is hindered by the uneven thickness of the nail plate and the unpredictable outcome of the procedure. The automatic debridement device aims to safely remove layers of the nail plate to achieve a surface conformal to the topography of the nail bed. This would result in a uniform thickness of the remaining nail, which would be conducive to laser treatment of nail fungus. The century-old paradigm of radiation treatment is to deliver therapeutic doses to the diseased tissue while preserving normal tissue. The uniform thickness of the nail plate could permit a conformal and uniform fungicidal dose delivery of laser radiation. The wavelength and intensity of the radiation could be selected such as to predictably deliver a safe and uniform dose distribution to the fungus-infected layers of the nail and nail bed while avoiding damage to healthy structures. Using the automatic debridement device with laser treatment is expected to significantly raise the cure rates of laser treatments.

The laser light transmission across the nail plate depends on the wavelength and the nail thickness. The amount of light capable of passing through the nail plate decreases exponentially with increasing thickness, and the exponential constant vary according to the wavelength of light employed. The light transmittance through the nail plate could be estimate according to equation 2 (Lambert-Beer's law), see FIG. 5:

$$T = \frac{I}{{I}_{0}} = {e}^{- β \cdot {h}_{b}} \qquad ( 02 )$$

where T is the Transmittance (the ratio of the transmitted light intensity in percentage),
h_{b} is the thickness of the nail plate 500, and
β is the exponent constant and it is a function of the light wavelength

A reduction of the barrier thickness from h_{b} to h_{bf} results in an increase of the light transmittance from T₁ to

$${T}_{2} = {T}_{1}^{\frac{{h}_{bf}}{{h}_{b 1}}} .$$

For example, at wavelength of 509 nm (β=2.612) the transmittance T₁ through a nail plate of thickness 1.8 mm is 2.17%. A reduction of the nail plate's thickness to 0.15 mm, results in transmittance of 72.7%.

In some embodiments, reduction of the nail plate thickness (i.e., the barrier) enable the use of low-power lasers to eradicate the infection on the nail, e.g., nail fungus.

In some embodiments, the camera 711 is utilized to detect the infection region of the nail, via the discoloration of the nail, wherein an infected nail will appear yellowish, white, or opaque. In some embodiments, the thickness sensor is utilized to detect the infected region of the nail via an increase in the nail plate thickness, which is typical of fungal nail infection.

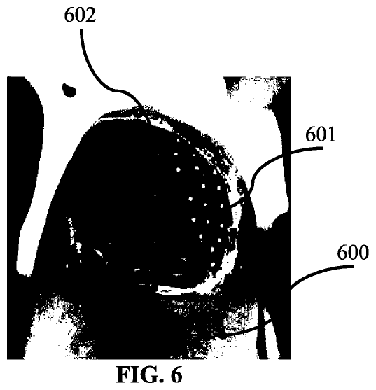

FIG. 6 is an exemplary depiction of a toenail 600 suffering from a common form of Onychomycosis (fungal infection) only in region of the nail 601. The infected region 601 is showing decolorization typical for Onychomycosis. The nail's region for treatment may be defined either manually or automatically by the controller system software. In some embodiments, the robotic debridement device drills a pattern and density of tiny holes across the defined region of the nail plate 602, as predetermined by the toolpath generated by the controller system software. In some embodiments, the size of the holes varies, e.g., near the edges of the nail the holes' size is reduced.

In some embodiments, the debridement and perforating are performed inside a bath filled with liquid, e.g., water. In some implementation, the liquid is cooled or heated to a predefined temperature, as defined by the control system. In some embodiments, said liquid bath assists in the removal of heat during the debridement and drilling operations, as well as, in the collection and disposal of any nail debris, fungi, bacteria, or other particles that are generated during the debridement or perforating of an infected nail.

### Device:

The present invention further features, in part, a device for the automated, robotic debridement of infected finger or toenails, e.g., infected with Onychomycosis. In some embodiments, said device perforates the infected nail with small holes such that a topical treatment can more easily penetrate the nail plate.

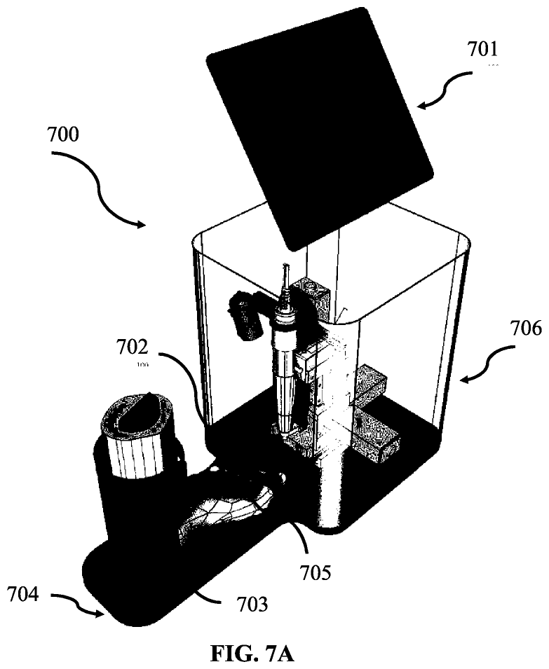

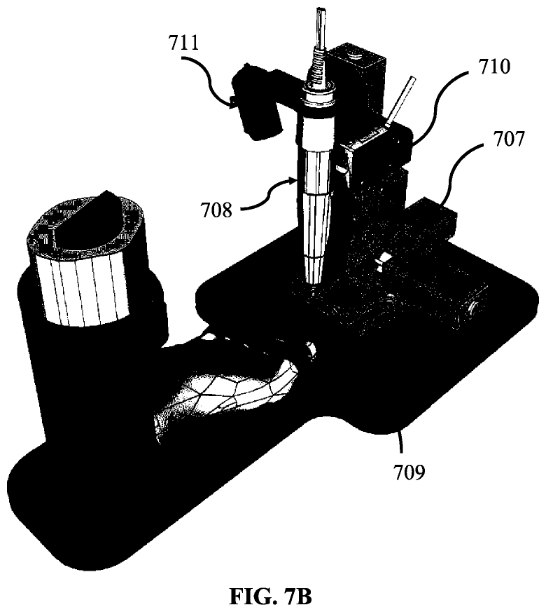

FIG. 7A and FIG. 7B are an illustration of an exemplary embodiment of the robotic debridement device 700. The device is designed to be compact, portable, and freestanding. It can be easily transported between rooms and placed on the floor, or a table, in a treatment room. Materials used to construct the device, especially inside the treatment volume, should have anti-microbial properties (e.g., 304 stainless steels). Plastic components, including the housing, should be made of medical-grade polymers.

Four height-adjustable, semi-rigid feet are attached to the bottom of the base 704. The feet protect the base from scratches and allow the device to be balanced on a surface that is not completely planar.

In some embodiments, said device 700 comprises: a base 704, a foot or hand holder 703 and 705, a multi axis motion stage 707, a debridement head 708, a debridement bit 709, a distance sensor 710, a thickness sensor, and a camera 711. In some embodiments, said device include a low-power laser.

In some embodiments, said base 704 of the robotic debridement and perforating device comprises a case 706 consisting of four side panels and a top cover.

In some embodiments, one such side panel has a hole cut out in the bottom center 702 such that the foot or hand holder is able to slide in and out of the case during the procedure. A door can cover the access port 702 when not used, or the part of the base 704 that protrudes outside the enclosure and provides stabilization for the treated foot might be mounted on a hinge and rotate 90 degrees to cover the port access.

In some embodiments, said top cover of the base 704 further comprises a touch screen computer monitor 701. In some embodiments, the touch screen 701 might be integrated into the device covers 706. Said touch screen monitor may be used to watch a real-time view of the debridement and perforating of the nail by the camera 711 of the device.

In some embodiments, said touch screen 701 is configured to display treatment options to the operator, and receives input guides from the operator.

During the debridement or perforating, nail debris, fungal, bacterial, and other particles are generated. In some embodiments, the device includes systems to collect and dispose of these particles. Examples of such system can include but are not limited to using vacuum to capture the particles and collect them into a replaceable HEPA filter bag.

In some embodiments, A UV lamp can be used for sanitizing the device between treatments. The application of Ultraviolet germicidal irradiation to disinfection has been an accepted practice since the mid-20th century. The UV short wavelengths allow the light to penetrate inside the bodies of pathogens to destroy them. Ultraviolet light can destroy pathogens which makes it incredibly effective on viruses, bacteria, and fungi. UV wavelengths within the spectrum 226-328 nm have been used for disinfection, 254 nm being a typical wavelength. These wavelengths can damage human skin and eyes, hence appropriate sealing of the device 700 during sterilization is required.

In some embodiments, said foot or hand holders 703 and 705 provide a way to secure and immobilize the entire foot or hand, as well as individual nails, during debridement. In some embodiments, said foot or hand holders 703 and 705 separates and fixes the orientation of each individual nail that is to be debride, using a reasonable force that would not make the patient uncomfortable. In some embodiments, said holder 703 is able to enter the device case via a cut-out entry port 702, through which the toe or fingers are individually positioned under the debridement head.

In some embodiments, said foot or hand holders 703 and 705 is designed to provide a maximum force equal or higher than the debridement force extract by the debridement bit on the nail. In some embodiments, said foot or hand holders 703 and 705 utilize a soft material to fix the toe or figure in place. The maximum debridement force extract by the debridement bit on the nail should not exceed a predefined threshold defined by the maximum allowed deflection of the soft material. In some embodiments, the path planning algorithm is designed to limit the extracted force between the debridement bit and the nail, e.g., limiting the acceleration of the debridement head. In some embodiments, the foot or hand holders 703 and 705 are designed to provide a different force based on the force extracted by the debridement bit on the nail. A force sensor on the foot or hand holders 703 and 705 or a force sensor in the debridement head can provide a measurement of this force. In dome embodiments, the foot or hand holders 703 and 705 are designed to provide an initial mechanical alignment between the foot or hand to the motion stage.

In some embodiments, the robotic debridement and perforating device further comprises a multi-axis motion stage (e.g., five-axis) 707 which is mounted onto the base of the device 704. The debridement head 708 is attached to the motion stage 707, such that the debridement head 708 can be moved in any direction and reach any required point with the required orientation on the nails. More specifically, an exemplary debridement bit 709 may be able to reach any point on each of the toe or fingernails that are in the necessary orientation.

In some embodiments, the distance sensor 710 or thickness sensor is mounted onto the motion stage 707. In some embodiments, data from the scan from the distance sensor is sent to the main controller, where the algorithm described here generates the toolpath for the debridement head to follow. In some embodiments, using a robotic debridement head and a computer-generated toolpath ensures optimal conformal debridement of the nail plate.

In some embodiments, the camera 711 is mounted on the motion stage 707 in such a way that generates clear images of the debridement process, which are then used to define specific areas on the nail which need debridement. Such an arrangement may enable the device to be able to scan one or more nails. An imaging processing algorithm might be used to enhance the image before projecting it on the touch screen 706 for the operator. The image captured by the camera could be utilize for generating motion limits for the toolpath, that can be used as another layer of safety and prevent damage to the surrounding tissues.

In some embodiments, the debridement head 708 is mounted on the motion stage 707. The debridement head has a variable speed starting at 1,000 rpm and up to, at-least, 15,000 rpm. Speeds above 20,000 rpm are usually used for acrylic nails, while speeds less than 10,000 rpm are for natural nails.

In some embodiments, the debridement bit 709 is attached to the debridement head 708. In some embodiments, the debridement bit 709 comes separate from the debridement head. In some embodiments, the debridement bit 709 should be replaced between treatments to prevent reinfection or cross-infection between treatments.

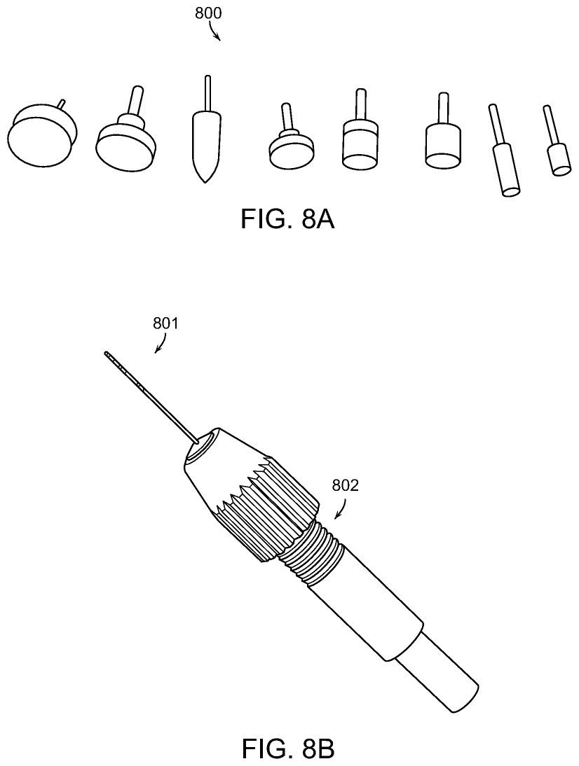

FIG. 8A is showing an example of an array of debridement bits 800. FIG. 8B is showing an example of a drilling bit 801 and a drilling bit holder 802. The debridement head can utilize any of these bit types. Some of the attachments bit included, but not limited to nail filer, nail buffer, cleaning brush, cone-shaped nail files, flat head nail files, rounded nail fail, and sanding bands of different grinding grades (e.g., grit #80, #120, #180). The drilling bit material included, but not limited to steel, titanium, and hard steel. The drilling bit size can include, but it is not limited to 0.1 mm, 0.2 mm, 0.3 mm, 0.4 mm, and 0.5 mm.

In some embodiments, the robotic debridement and perforating device may include an array debridement 800 or drilling bits 801 for the debridement and perforating, which are interchangeable depending on the need of the user. In some embodiments, the replacement of a bit is done manually. In some embodiments, the device includes an automatic bit exchanger.

In some embodiments, the contact between the debridement 800 or drilling bits 801 and the nail plate defines the debridement surface. A precise calibration between this surface and the distance sensor 710 is necessary for building the toolpath. This can be accomplished, for example, by using a mechanical touch sensor that registers the precise location of the debridement bit, wherein said touch sensor is located below the debridement head. In some embodiments, the calibration process includes moving the debridement bit above the touch sensor, and slowly moving it using the vertical axis of the motion stage until a contact is detected. The reading from the distance sensor 710, at this location, is registered in the computer system memory and used to generate the toolpath and parameters for such.

In some embodiments, the foot or hand holder 703 might also include a first electrode of a resistance sensing circuit. the second electrode is attached to the debridement bit 709. The circuit can be utilized to immediately withdraw and stop the debridement bit rotation if the measured impedance is different than expected.

In some embodiments, pressing the emergency button located on the device case 706 cuts the power to the system immediately, and the debridement bit is automatically withdrawn from the nail surface. In some embodiments, pressing the emergency button starts a shutting down process comprising the following steps: 1) the debridement bit is withdrawn from the nail; 2) the rotation is actively stopped without coast-down; And 3) power to the device is shut off

In some embodiments, the contact force between the debridement bit 709 and the nail surface is controlled or limited. In some embodiments, said contact force determines the surface quality because excessive contact force can lead to over debridement and too little contact force will lead to under debridement. In addition, an abrupt increase in this force may cause pain or tissue damage and should be prevented. In some embodiments, a passive force control is realized using an elastic material (e.g., spring). In some embodiments, advantages of use of a passive force control are that it is simple and has a fast response rate. In some embodiments, disadvantages of use of a passive force control are that it is difficult to achieve accurate force control and it can only provide small adjustments of the force.

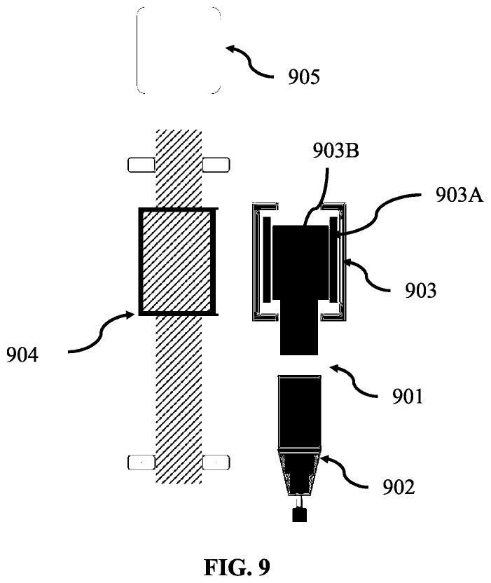

In some embodiments, an active force control is realized using a force sensor. Active force control may accurately and dynamically adjust the contact force with high bandwidth. FIG. 9 depicts an exemplary of a force sensor 901 mounted between the drill bit holder 902 and the linear motor 903. The linear motor is mounted on the vertical axis 904 of the motion stage. In some embodiments, the linear motor is a voice coil motor. The high acceleration of the voice coil motor is required for a high bandwidth force control. The voice coil motor is composed of a shaft with permanent magnet 903B, and a coil 903A. The vertical axis is a composed of a ball screw 904 driven by a stepper or servo motor 905. In some embodiments, the motion stage 707 is responsible for the path tracking and the posture of the drill bit. The contact force control is performed by the additional high bandwidth linear motor and force sensor system. The contact force control is separated from the motion stage control.

### Computer and Control Systems, Software and Algorithm:

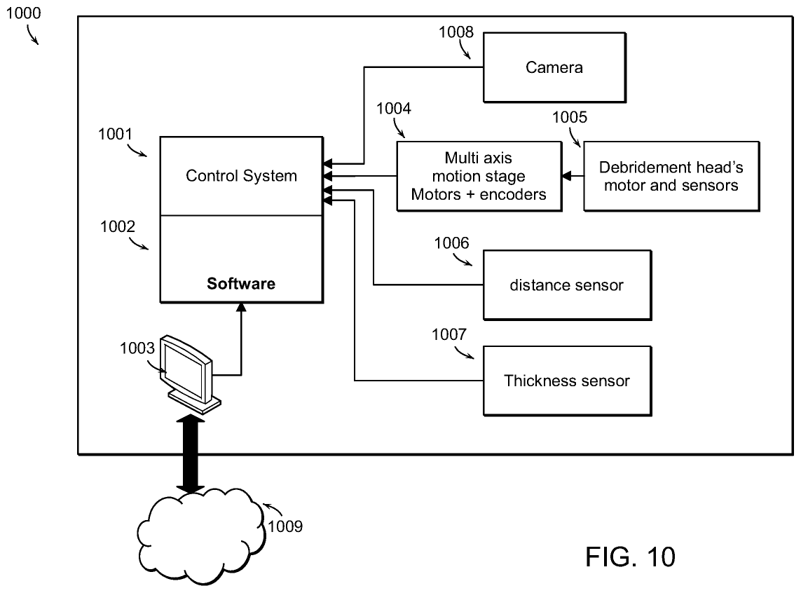

FIG. 10 is an exemplary depiction of an embodiment of the robotic debridement device computer and control system 1000. wherein said systems include a computer system with a screen 903 wherein said screen is located on the top of the device and is used by the operator to manage the control and software systems, a control system 1001 wherein said control system dictates the debridement toolpath and other software programs being run 1002. In some embodiments, the physical components of the device are linked and communicating with main controller 1001, e.g., the multi axis motion stage 1004, the debridement tool 1005, the distance sensor 1006, the thickness sensor 1007, and the camera 1008.

The computer system 1003 is programmed to implement methods for robotic debridement and perforating of nails. In some embodiments, the computer system 1003 can be remotely located with respect to the robotic device.

The computer system 1003 includes a central processing unit (CPU) which can be a single or multi core processor, or a plurality of processors for parallel processing. The computer system 1003 also includes memory (e.g., random-access memory, read-only memory, flash memory), electronic storage (e.g., hard drive), communication interface (e.g., network adapter) for communicating with one or more other systems, and peripheral devices, such as, electronic display adapters, data storage, and other memory. The computer system 1003 can be coupled to a computer network (“network”) 1009 with the aid of a communication interface. An example of the network can be, but it is not limited to, the Internet. In some embodiments, the network can include one or more computer servers. In some embodiments, the network can implement a peer-to-peer network, which may enable devices coupled to the computer system to behave as a client and server.

The CPU inside said computer system 1003 can execute a sequence of machine-readable instructions, which can ne embodied in a program or software. The instructions may be stored in a memory. The instructions can be directed to the CPU, which can subsequently program or otherwise configure the CPU to implement methods of the present disclosure.

The CPU can be part of a circuit, such as an integrated circuit. One or more other components of the system 1000 can be included in the circuit. In some cases, the circuit is an application specific integrated circuit (ASIC).

The said storage unit can store files, such as drivers, libraries, and saved programs. The storage unit can store data, e.g., user preferences and user programs. The computer system 1000 in some cases can include one or more additional data storage units that are external to the computer system 1003, such as located in the control system or on a remote server that is in communication with the computer system 1003.

The computer system 1003 can communicate with one or more remote computer systems through the network. For example, the remote computer can be, but is not limited to, tablet PC's (e.g., Apple iPad, Samasung Galaxy Tab), cellphones (e.g., Apple iPhone, Android-enable device).

Methods and algorithms describe herein can be implemented by way of machine executable code stored on an electronic memory of the computer system 1003 or control system 1001. The machine executable or machine-readable code can be provided in the form of a software. During use, the code can be executed by the CPU. In some cases, the code can be retrieved from the storage unit and stored on the memory for ready access by the processor.

The code can be pre-compiled and configured for use with a machine having a processer adapted to execute the code or can be compiled during runtime. The code can be supplied in a programing language that can be selected to enable the code to execute in a pre-compiled or as-compiled fashion.

In some embodiments, the communication between the main controller 1001 and the different components is based on different field bases topologies (e.g., Ethercat).

In some embodiments, the software 1002 is connected to the control system 1001 and monitor. The software controls the system and presents the status and sensor readings on the screen.

In some embodiments, the control system 1001 includes, but not limited to a main controller and several single-axis motion controllers. The motion stage axes, as well as other axes in the device, might be driven by servo or stepper motors. The main controller synchronizes the motions of the different axes, according to defined toolpath.

The said single-axis motion controllers can drive the motors using a closed-loop speed, position, or force control, e.g., dual speed and position closed-loop control. Controlling the axes is based on the encoder readings from each axis.

In some embodiments, the camera 711 or the distance sensor 710 is used to stop the operation of the machine when the hand or foot has moved, by detecting a change in distance or angle of the nail to the sensor.

In some embodiments, where a drilling bit is used, a predefined pattern and density of tiny holes across the nail surface are automatically drilled. In some embodiments, the controller system utilizes the nail plate thickness data collected from the thickness sensor previously to determine the distance at which to stop drilling and pull back once the drilling bit has broken through the underside of the nail plate so as to prevent drilling into underlying tissue.

In some embodiments, the resistance force exerted on the drilling bit by the nail plate can be measured by the motor current of the vertical axis motor 903 or the force sensor 901. In some embodiments, where the drill bit has broken the underside of the nail plate, the motor current or the measured force should abruptly drop, and this change in the motor current or measured force is used instead of the thickness sensor to stop the drilling and pull back the drilling bit. In some embodiments, the nail's thickness at the drilling bit location is measured by registering the distance traveled by the vertical axis 904 from the drilling starting point until the point where the drill bit has broken the underside of the nail plate.

In some embodiments, if enough measurements are collected, the thickness measurements from the vertical axis motor 903 current can replace the information obtained by a thickness sensor 710.

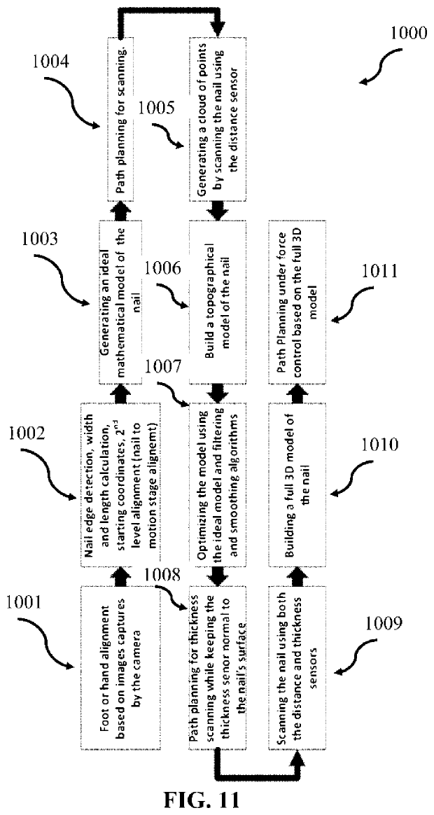

FIG. 11 is an exemplary depiction of path planning algorithm based on surface and thickness measurements, for the automatic robotic conformal debridement under force control of an infected nail. In some embodiments the targets of the path planning are: 1) reduce the nail thickness to its structural but safe limit, which reduced the barrier for topical drug diffusion through the nail plate, reduces the thermal barrier for laser and other therapeutics, and increase the transmittance of lasers and other forms of radiation through the nail plate. 2) achieving uniform nail thickness, which increases the effectiveness of radiation treatments. 3) achieving high nail surface quality, eliminate scratches and other surface related defects, which improve the cosmetic appearance of the nail. 4) minizine the treatment time. Embodiments of the path planning algorithm are discussed in detail below with respect to treatment of nail fungus. However, the disclosed embodiments can be employed for other applications without limitation.

In some embodiments, the foot or hand holders 703 and 705 are designed to provide an initial mechanical alignment between the foot or hand to the motion stage. The second level of alignment between the foot or hand and the motion stage is based on images of the foot or hand capture by the camera 711 as shown in the first step of the path planning algorithm 1101.

In some embodiments, the second step of the path planning algorithm 1102 provides for final level of alignment between the nail and the motion stage, based on images captures by the camera 711, wherein the nail's width and length are calculated. In some embodiments, the starting coordinate for the debridement procedure is also defined in this step.

In some embodiments, the third step of the path planning algorithm 1103, comprises building an ideal mathematical model (equation) of the nail based on the length and width measurements from the previous step.

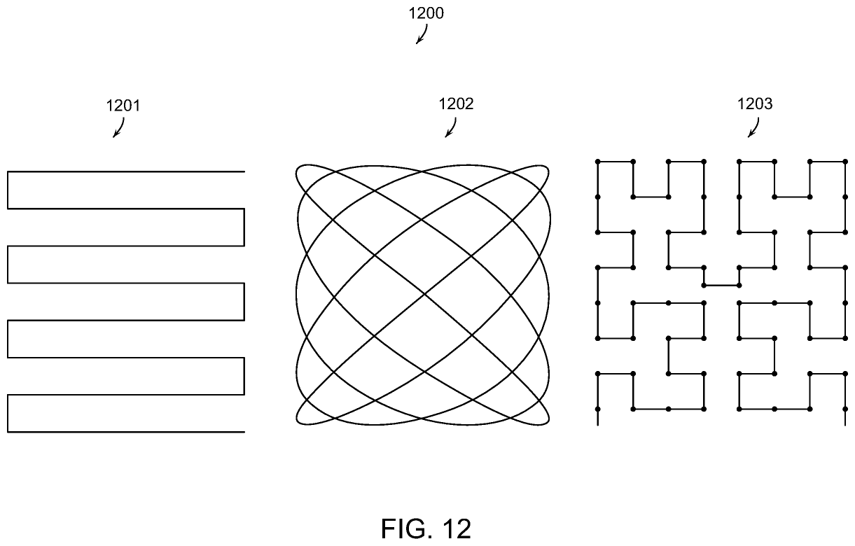

In some embodiments, the fourth step of the path planning algorithm 1104 comprises generating the tool paths for the distance sensor scanning step 1005. FIG. 12 depicts an exemplary of three possible two-dimensional plane-filling tool paths patterns, including but not limited to, scanning paths 1201, Lissajous paths 1202, and Peano paths 1203. The algorithm automatically defines the optimal tool-path parameters based on the ideal mathematical model from step 1103, the distance sensor's properties, and the tool-path patterns being used.

In some embodiments, the fifth step 1105 comprises scanning the nail using the distance sensor 710 based on the toolpath from step four, resulting in a cloud of points representing the surface of the nail.

In some embodiments, the sixth step 1106 comprises building a topographical model of the nail from the cloud of points from the previous step 1105.

In some embodiments, the seventh step 1107 comprises, the algorithm improving the topographical model by implementing different filtering and smoothing techniques. In some embodiments, the algorithm optimizes the topographical model by using the ideal mathematical model of the nail.

In some embodiments, the eight step 1108 comprises, the algorithm generating the tool paths for the thickness sensor scanning step 1109. In some embodiments, for accurate thickness measurements it is required that the thickness sensor is always kept normal to the surface of the nail. The algorithm generates a tool path that uses one of the patterns 1200, with an additional rotational parameter per each scanning point.

In some embodiments, the ninth step 1109 comprises scanning the nail using both the distance sensor 710 and the thickness sensors.

In some embodiments, In the tenth step 1110 comprises, the algorithm building a full 3D model of the nail, e.g., the outside surface of the nail's model is represented by equation 3 (two-parameters surface model [symbol] ²→ [symbol] ³):

z=f(x,y) (03)

In some embodiments, the eleventh step 1111 comprises, generating a toolpath under force control based on the full 3D model from the previous step 1110. This tool path is used by the robotic debridement device to perform the debridement procedure. The algorithm uses an indirect path generation approach, wherein the algorithm builds paths on a two-dimensional parametric plan using one of the said patterns 1200, then mapping the paths on to the three-dimensional surface, represented by equation 3, extracted from the full 3D model created for the nail. This process then maybe repeated for each debridement layer.

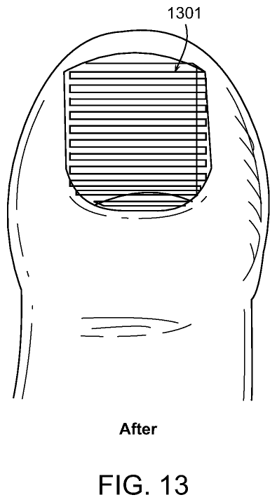

FIG. 13 is an exemplary depiction of mapping the two-dimensional scanning path pattern 1201 as described on to the nail's three-dimensional surface 1301, using the model represented by equation 3.

In some embodiments, the tool path parameters include, but are not limited to, line spacing, pitch, feed rate, rotational speed. In some embodiments, these parameters are optimized using design of experiment (DOE) methods (e.g., full factorial design).

In some embodiments, two separate control systems are implemented, wherein the motion stage control system is responsible for path tracing (XYZ) and keeping the debridement bit 709 normal to the nail's surface using the rotational DOF.

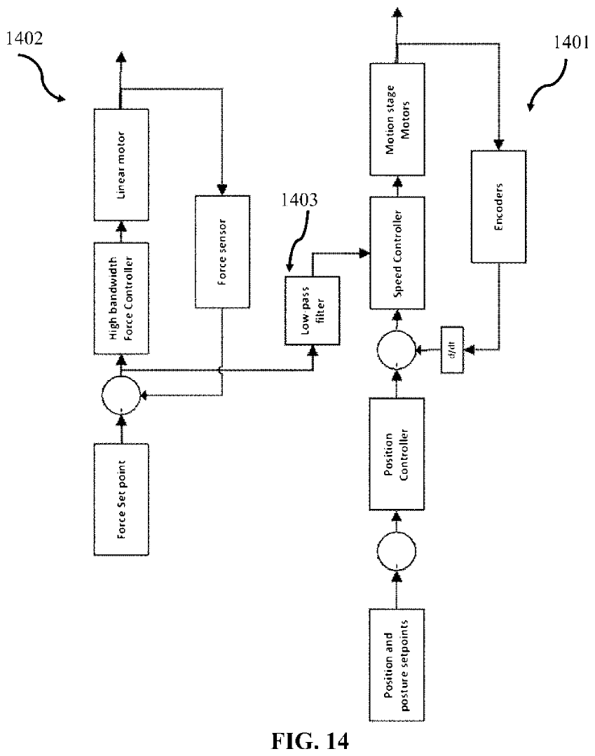

FIG. 14 depicts an exemplary of the control system. In some embodiments, the motion stage control system is a dual position and velocity closed-loop control system 1401. In some embodiments, the contact force is controlled by a separate system 1402. An example of possible motion and force controllers 1401 and 1402 can include but is not limited to a PID controller.

In some embodiments, the force sensor signal 1403 is utilized inside the motion stage controller 1401 for low frequency adjustment of the motion stage speed. If the force error, the difference between the actual and force setpoint is zero, there is no impact on the speed controller. If the error is not zero, the controller changes the motion stage speed to allow more or less time for the contact force control system to adjust the contact force.

In some embodiments, the control system is connected to a low-power laser. In some embodiments, the information from the debridement and perforating of the nail is used to optimize the laser treatment, such as for optimizing the wavelength and intensity of the laser, resulting in an exact delivery of therapeutic radiation doses to the diseased tissues while preserving the normal tissues. In some embodiments, the control system shuts down or reduces the laser intensity around the locations of the drilled holes.

In some embodiments, the software collects information during the treatment. Example of information that can be collected, but is not limited to the camera images before, after and during the treatment, the measurements from the distance or thickness sensors, and the generated 3D model of the nail.

In some embodiments the information collected is stored locally in the computer system 1000, or in the cloud 1009.

In some embodiments, said collected information is later used for analyzing treatment progress. In some embodiments, a comprehensive treatment progress report, that could be shared with the patient, is generated based on said collected data. In some embodiments, said software 1002 provides treatment recommendations which are based on the historical data collected during the treatments. Examples of treatment recommendations may be residual nail plate thickness, drilling patterns, debridement speed, and toolpath parameters.

In some embodiments, information collected from many patients is used to improve the overall efficacy of the treatment.

What is claimed is:

1. A robotic nail debridement system, the system comprising:
a motion stage;
a foot holder or hand holder configured to align a subject's hand or foot in relation to the motion stage;
a debridement head mounted on the motion stage;
one or more cameras and or sensors configured for imaging and/or sensing one or more nails on the subject's hand or foot when the subject's hand or foot is aligned in relation to the motion stage by the foot holder or hand holder; and
a controller system in communication with the motion stage, the debridement head, and the one or more cameras and or sensors;
wherein the controller system comprises a software program executable by the controller system.

2. The system of claim 1, further comprising
a case comprising a port, wherein the port is aligned with the holder such that the foot or hand being secured by holder enters the case such that the nail is aligned under the debridement head.

3. The system of claim 2, wherein the holder is configured to secure and immobilize individual digits of the subject's hand or foot in relation to the motion stage.

4. The system of claim 1, wherein the one or more sensors comprise one or more distance sensors and/or thickness sensors.

5. The system of claim 1, comprising a debridement bit mounted in the debridement head.

6. The system of claim 1, wherein the one or more cameras are mounted on the motion stage.

7. The system of claim 1, wherein the one or more sensors are mounted on the motion stage.

8. The system of claim 1, wherein the software program is configured to automatically generate a toolpath of the debridement head based on the imaging and/or sensing of the one or more nails.

9. The system of claim 8, wherein the debridement head is configured to follow the toolpath.

10. The system of claim 8, wherein the toolpath enables the debridement head to perform automated debridement of the one or more nails.

11. The system of claim 8, wherein the toolpath is configured to for the debridement head to reduce an infected nail thickness to a safe structural limit and achieve uniform nail thickness across the nail.

12. The system of claim 8, wherein the toolpath enables the debridement head to perform automated perforation of the one or more nails with small holes.

13. The system of claim 8, wherein the software program is configured to generate a respective toolpath for each nail on the subject's hand or foot.

14. The system of claim 8, comprising an automatic bit exchanger configured to select and install a bit on the debridement head wherein the bit is selected from an array of debriding bits and drilling bits in accordance with the toolpath.

15. The system of claim 1, comprising a force sensor configured to measure a force exerted by the debridement bit onto the nail surface.

16. The system of claim 15, comprising a linear motor mounted on a vertical axis of the motion stage.

17. The system of claim 16, wherein the force sensor is mounted between a bit holder portion of the debridement head and the linear motor.

18. The system of claim 8, wherein the toolpath includes force parameters controlling a force exerted by the debridement head on the one or more nails.

19. The system of claim 18, wherein the force parameters in the toolpath are updated in real time based on the imaging and/or scanning of the one or more nails.

20. The system of claim 18, wherein the force parameters in the toolpath are configured to achieve uniform thickness of the one or more nails.

21. The system of claim 15, comprising a closed loop force control system configured for controlling force applied by a debridement bit and/or a drill bit on the one or more nails, wherein the closed loop force control system includes the force sensor.

22. The system of claim 15, wherein the holder comprises a force adjustment system configured to adjust a force exerted by the holder onto the digits to match the force exerted by the debridement bit as determined by the force sensor.

23. The system of claim 16, wherein the linear motor comprises a voice coil motor comprising a shaft, a permanent magnet and a coil.

24. The system of claim 16, wherein the vertical axis comprises a ball screw driven by a stepper motor or a servo motor.

25. The system of claim 1, wherein the controller system is configured to collect data from the sensors and/or the camera.

26. The system of claim 25, wherein the data includes images of a nail and scans of the thickness of the nail before and after the debriding and/or perforating procedure.

27. The system of claim 25, wherein, the software program is configured to recommend new treatment parameters based on the data.

28. The system of claim 27 wherein the new treatment parameters are in the group consisting of required nail thickness, debriding patterns, drilling patterns, and perforation locations.

29. The system of claim 1, comprising a debris collection system configured to capture and collect the nail debris, fungal, bacterial, and other particles that are generated during the debridement and/or perforating procedure.

30. The system of claim 29, wherein the debris collection system comprises a vacuum component and a replaceable HEPA filter bag.

31. A method, comprising:
securing a hand or foot of a subject in a foot holder or hand holder of a robotic debridement system, wherein the robotic debridement system comprises
a motion stage,
the foot holder or hand holder configured to align a subject's hand or foot in relation to the motion stage,
a debridement head mounted on the motion stage;
one or more cameras and or sensors configured for imaging and/or sensing one or more nails on the subject's hand or foot 711 when the subject's hand or foot is aligned in relation to the motion stage by the foot holder or hand holder, and
a controller system in communication with the motion stage, the debridement head, and the one or more cameras and or sensors;
aligning one or more nails of the hand or foot under the debridement head; and
controlling the debridement head by the controller system to follow a toolpath for debriding the one or more nails.

32. The method of claim 31, comprising mounting a debridement bit in the debridement head.

33. The method of claim 31, comprising:
mounting a drilling bit in the debridement head; and
controlling the debridement head by the controller system to follow a toolpath for perforating the one or more nails.

34. The method of claim 33, comprising drilling a pattern of holes across an infected region of the one or more nails, wherein the pattern is defined by the toolpath.

35. The method of claim 34, comprising applying a topical antifungal treatment to the one or more nails after drilling the pattern of holes.

36. The method of claim 35, comprising applying the topical antifungal treatment to the pattern of holes such that the treatment directly contacts infected layers of the one or more nails.

37. The method of claim 31, comprising:
attaching a low power laser to the motion stage; and
applying low power laser irradiation from the low power laser to the one or more nails.

38. The method of claim 31, comprising:
imaging and/or scanning the one or more nails with the one or more cameras and or sensors wherein the imaging and/or scanning is controlled by the controller system; and
automatically generating a toolpath of the debridement head for the nail based on the imaging and/or scanning.

39. The method of claim 38, wherein the toolpath is generated by the controller system to reduce nail thickness of the one or more nails to a safe structural limit and to achieve uniform nail thickness across the each of the one or more nails.

40. The method of claim 38, wherein the toolpath is generated by the controller system to remove scratches and/or surface defects from the one or more nails.

41. The method of claim 38, wherein the toolpath is generated to improve cosmetic appearance of the one or more nails.

42. The method of claim 38 comprising:
acquiring distance and thickness data for the one or more nails from the one or more sensors;
generating a topographical map of the one or more nails based on the distance and thickness data; and
generating a 3D model of the one or more nails based on the topographical map.

43. The method of claim 42, comprising:
generating the toolpath for the robotic debridement device based on the 3D model.

44. The method of claim 31, comprising:
capturing images of the infected nail by the one or more cameras; and
defining motion limits of the toolpath based on the images.

45. The method of claim 44, comprising:
defining infected regions of the one or more nails based on discoloration in the images; and
defining the motion limits based on the infected regions.

46. The method of claim 31, comprising displaying real-time images of the foot and the debridement head as the debridement head follows the toolpath, wherein the images are acquired by the one or more cameras.

47. The method of claim 31, comprising:
detecting a change in distance or angle of the nail by the one or more sensors and/or cameras; and
stopping motion of the robotic debridement system in response to detecting the change.

48. The method of claim 38, comprising:
generating a first portion of the toolpath for debriding the one or more nails with a debriding bit; and
generating a second portion of the toolpath for perforating the one or more nails with a drill bit.

49. The method of claim 48, comprising replacing the debriding bit in the debridement head with a drill bit between the first portion of the toolpath and the second portion of the toolpath.

50. The method of claim 38, comprising:
stopping perforation of the one or more nails and retracting the debridement head when a drill bit installed in the debridement head has broken through the underside of the nail plate; and
moving the debridement head to a next location to be drilled on the toolpath.

51. The method of claim 31, comprising applying UV radiation to the robotic debridement system, for sanitizing the robotic debridement system between uses, wherein the UV radiation has a wavelength of approximately 225 nm-330 nm.
